(19) United States
(12) Patent Application Publication
(10) Pub. No.: US 2013/0267925 A1
(43) Pub. Date: Oct. 10, 2013

(54) DUAL PURPOSE ADHESIVE BANDAGE

(71) Applicants: Héctor M. Aybar López, Cottage Grove, MN (US); Wayne K. Dunshee, Maplewood, MN (US)

(72) Inventors: Héctor M. Aybar López, Cottage Grove, MN (US); Wayne K. Dunshee, Maplewood, MN (US)

(21) Appl. No.: 13/996,339

(22) PCT Filed: Dec. 21, 2011
(86) PCT No.: PCT/US11/66583

§ 371 Date: Jun. 20, 2013

Related U.S. Application Data
(60) Provisional application No. 61/426,324, filed on Dec. 22, 2010.

(51) Int. Cl. A61F 7/10; A61F 13/02

(52) U.S. Cl. CPC A61F 7/10; A61F 13/0206
USPC 604/377; 607/112; 604/385.03

(57) ABSTRACT

Dual purpose adhesive bandages comprise a backing, a pressure sensitive adhesive disposed on the backing, and an absorbent pad adhered to the backing. The absorbent pad is configured so as to form a pouch having at least one open end for releasably receiving a thermal pack. Thermal management kits comprise the dual purpose bandage and a thermal pack.

### FIELD

This invention relates to medical adhesive bandages that can also hold a thermal pack and to kits containing the medical adhesive bandages and a thermal pack.

### BACKGROUND

Pressure sensitive medical tapes and bandages are widely available. Medical adhesive bandages are used to cover cuts, scrapes, and other skin conditions or wounds. Medical adhesive bandages typically include a backing, an absorbent pad, and a pressure sensitive adhesive to keep the bandage in place.

Therapy devices comprising a material that can be used as a hot or cold compress are also widely available. Such devices, which are commonly referred to as hot or cold packs, typically comprise a sealed envelope with a material inside such as a thermal gel that can be used for application as desired of either heat or cold (that is, the material operates as a heat source or a heat sink, respectively).

Hot and cold packs are usually held in place by the patient receiving treatment. This can be inconvenient for the patient. Therefore, hot and cold packs that adhere to the skin have been designed. For example, U.S. Pat. No. 3,871,376 (Kozak) describes an adhesive bandage that is a combination absorbent dressing and flexible cooling device; U.S. Pat. No. 5,887,437 (Maxim) describes a self-adhering cold pack; and WO 03/059219 describes an adhesive bandage that has a cold pack adhered between a gauze pad and the outer layer of the bandage. In each of these examples, the thermal pack cannot be removed from the bandage.

### SUMMARY

In view of the foregoing, we recognize that it would be advantageous to have an adhesive bandage with a thermal therapy device (for example, a hot or cold pack) that is easily removable.

Briefly, in one aspect, the present disclosure provides dual purpose adhesive bandages. The bandages comprise a backing, a pressure sensitive adhesive disposed on the backing, and an absorbent pad adhered to the backing by the pressure sensitive adhesive. The absorbent pad is configured so as to form a pouch having at least one open end for releasably receiving a thermal pack.

In another aspect, the present disclosure provides thermal management kits comprising the adhesive bandage of the invention and a thermal pack comprising a flexible sealed envelope and a thermal agent within the envelope.

The adhesive bandages of the disclosure can be used to hold thermal packs in place. The thermal pack can be easily removed from the bandage. This allows the user to place another thermal pack into the bandage without removing the bandage from their skin. For example, the user may remove a first cold pack from the bandage (without removing the bandage) after the cold pack has become to warm to be therapeutically effective and replace it with a second cold pack. The first cold pack may be placed back into a freezer and then used again at a later time.

Although the adhesive bandages of the present disclosure are configured to hold a thermal pack, they can still be used as regular medical adhesive bandages. Like conventional medical adhesive bandages, the bandages of the present disclosure have absorbent qualities while still providing a non-stick surface for a wound. The adhesive bandages thus serve dual purposes. They may be used as a conventional adhesive bandage or they may be used to hold a thermal pack in place. In addition, the adhesive bandages of the present disclosure advantageously allow the user to thermally treat a wound without placing a separate absorbent layer between the thermal pack and an open wound.

The adhesive bandages may be assembled, converted, and packaged on existing manufacturing lines that make conventional adhesive bandages with minimal changes.

As used herein, “adhesive bandages” or “bandages” will be understood to refer to medical adhesive bandages.

### BRIEF DESCRIPTION OF THE DRAWINGS

FIG. 1 is an adhesive bandage according to an embodiment of the present disclosure.

FIG. 2 is an adhesive bandage further comprising a removable lining.

FIG. 3 is a thermal management kit of the invention.

FIG. 4 is a thermal management kit of the invention in use.

### DETAILED DESCRIPTION

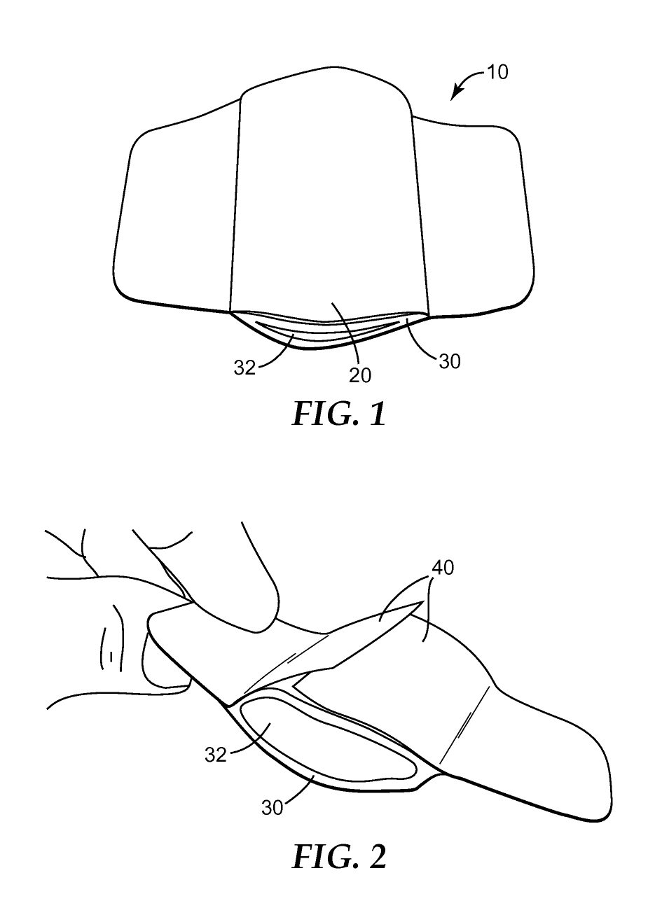

One embodiment of a bandage 10 (see FIGS. 1 & 2) of the present disclosure includes a backing 20, a pressure sensitive adhesive on a surface of the backing, and an absorbent pad 30.

### Backing

A wide variety of materials may be used to form the backing 20. The backing 20 can be tearable or nontearable, elastic or inelastic, stretchable or nonstretchable, porous or nonporous. Backings can be in the form of single or multilayer films, nonwoven films, porous films, foam-like films, and combinations of the foregoing. Backings can also be prepared from filled materials such as, for example, filled films (e.g., calcium carbonate filled polyolefins).

Film backing can be made by any known method of film forming such as, for example, extrusion, coextrusion, solvent casting, foaming, nonwoven technology, and the like. A backing 20 can have a wide variety of thicknesses so long as it possesses sufficient integrity to be processable.

Webs made from natural or synthetic fibers of mixtures thereof can be used. Woven or nonwoven materials can be employed. Melt-blown or spunbond techniques can be employed to make nonwoven webs.

In embodiments wherein the backing 20 comprises a laminate, there may be one or more additional layers, which can be a breathable, liquid impervious film. Typically this film is the outermost (i.e., top) layer. Examples of film materials include polyurethanes, polyolefins, metallocene polyolefins, polyesters, polyamides, polyether esters, and A-B-A block copolymers such as KRATON copolymers available from Kraton Polymers. In some embodiments, the outermost layer is a film that is substantially impervious to fluids such as could arise from the external environment, yet permit passage of moisture vapor such that the adhesive bandage is breathable.

The backing 20 can optionally include fibers, which may be absorbent or nonabsorbent, and typically are non-water absorptive. The fiber structures useful in the backing can include a multilayer configuration, a coated configuration, and a solid homogeneous configuration. Suitable multilayer fibers preferably have cores and outer layers composed of one or more polymers selected from polyolefins, polyesters, polyamides, and polyurethanes. Suitable coated fibers preferably have cores made of these polymers with coating covalently bonded, embedded, or adhered thereto. The homogeneous fibers are preferably made of any of the polymers listed above. Such fibers can be formed into backings using known weaving, knitting, or nonwoven techniques. Suitable backings including fibers are disclosed, for example, in U.S. Pat. No. 5,613,942 (Lucast et al.) and U.S. Pat. No. 6,083,856 (Joseph et al.) and WO 99/40952.

The backing can also optionally include a reinforcing scrim as disclosed in U.S. Patent Application Pub. No. 2007/0010777 (Dunshee et al.).

### Pressure Sensitive Adhesive

Suitable pressure sensitive adhesives that are useful in the adhesive bandages of the disclosure are known to be useful for application to skin. A suitable class of adhesives is disclosed, for example, in U.S. Pat. No. 6,441,092 (Gieselman). One example is a blend of 85 weight percent of 2-ethylhexyl acrylate/acrylic acid/ABP (96.5/3.5/0.05 weight ratio) and 15 weight percent Avalure AC 210 acrylate copolymer. Adhesives containing from about 5 to about 20 weight percent of such hydrophilic materials provide a good balance of desired moisture permeability without unduly softening the adhesive layer to yield undesirable levels of residue.

Other illustrative examples of useful adhesives include those described in U.S. Pat. No. 4,112,177 (Salditt et al.), particularly the tackified acrylate “skin layer adhesive” described in Example 1; U.S. Pat. No. 5,648,166 (Dunshee), RE 24,906 (Ulrich); U.S. Pat. No. 4,737,410 (Kantner) (see example 31); U.S. Pat. No. 3,389,827 (Abere et al.); U.S. Pat. No. 4,112,213 (Waldman); U.S. Pat. No. 4,310,509 (Berglund et al.); U.S. Pat. No. 4,323,557 (Rosso); U.S. Pat. No. 6,083,856 (Joseph et al.); and U.S. Pat. No. 6,497,949 (Hyde et al.); and U.S. Patent Application Pub. Nos. 2002/0165477 (Dunshee) and 2002/0193724 (Stebbings et al.).

The pressure sensitive adhesive preferably transmits moisture vapor to increase patient comfort. While moisture vapor transmission can be achieved through the selection of an appropriate adhesive, it is also contemplated that other methods of achieving a high relative rate of moisture vapor transmission may be used such as pattern coating the adhesive on the backing.

In preferred embodiments, the choice of adhesive is limited to those that are safe to use on human skin and preferably to those that are of the class known as “hypoallergenic” adhesives. Acrylate copolymers are adhesives of this class.

The thickness of the adhesive layer is generally about the same as is typically employed in medical adhesive bandages and will vary depending upon the backing and the pressure sensitive adhesive utilized. The thickness of the adhesive layer can be, for example, as little as about 12 microns when a thin backing is employed or as much as 100 microns or more when a thick backing is employed. The surface area needed will depend upon the strength of the pressure sensitive adhesive.

The adhesive layer can be provided on the backing using methods known in the art.

### Absorbent Pad

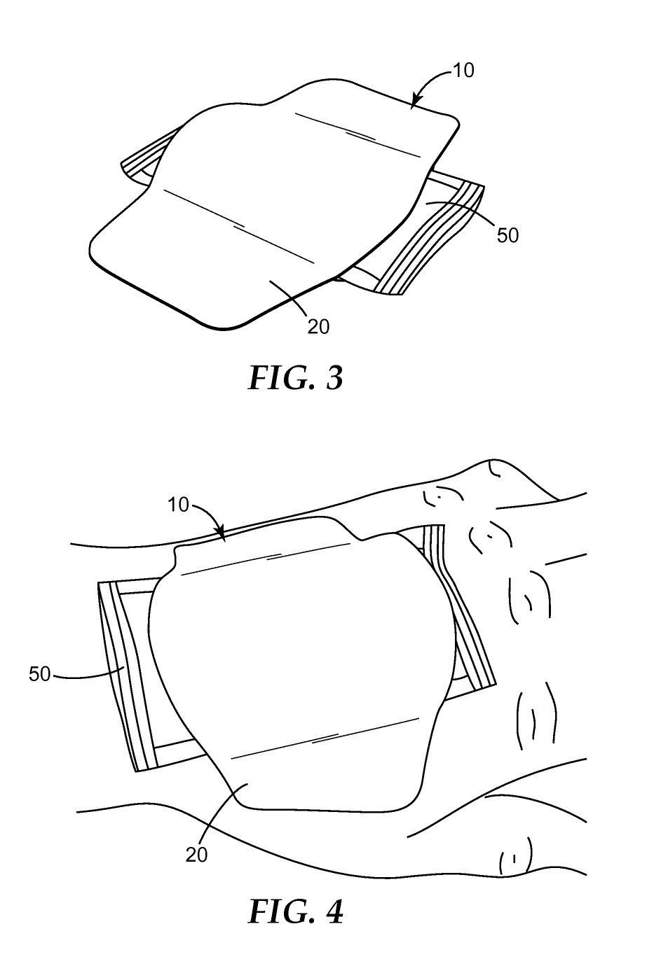

The adhesive bandages of the disclosure comprise an absorbent pad 30. The pad 30 is configured so as to form a pouch or pocket 32 having at least one open end for releasably receiving a thermal pack 50 (See FIGS. 3 & 4). Any useful configuration may be employed as long as the pad 30 securely holds the thermal pack. Preferably, the pad 30 is folded so as to form a tube (i.e., there are two open ends) into which the thermal pack can be slid. For example, the pad 30 may be in the form of a “c-fold”.

Typically, the pad 30 is provided such that the overall shape of the pad 30 (i.e., the perimeter of the pad 30) is generally rectangular shaped and is smaller than the overall dimensions of the backing It is typically centered on the inner major surface of the backing 20. The pad 30 may reach to one or both sides of the backing 20. Preferably it reaches to both sides of the backing 20. It will be understood that although the pad 30 is generally rectangular and centered on the backing 20, it can take any appropriate shape and/or the pad can be located off-center on the backing 20 as desired. The pad 30 may be coupled to the backing via the adhesive layer or by other methods known in the art.

The pad 30 may be made of any absorbent material that has sufficient stiffness to open up the tunnel or pocket for insertion of the thermal pack and sufficient wet strength. Preferably, the pad 30 is thin enough that it does not provide too much insulation. In some embodiments, the pad 30 has a minimum fabric weight of between about 1 and about 1.5 oz/yd², which is then folded over to double the fabric.

Examples of suitable pad materials include cotton gauze laminated to netting and nonwovens. Many nonwoven materials have sufficient stiffness for use in the adhesive bandages of the disclosure. Some nonwovens (e.g., 45 g/m² towel paper) and other materials such as cotton gauze, however, need to be stiffened, for example, with netting. Any netting that is non-absorbent and maintains its strength when wet may be utilized. Some useful netting fabrics are made, for example, from thermoplastics such as high density polyethylene (HDPE). Suitable netting fabrics are available from DelStar Technologies, Inc. under the Delnet® tradename. Useful laminate pads are also available from DelStar under the Stratex™ tradename.

### Removable Lining

As depicted in FIG. 2, the adhesive bandages of the present disclosure can be provided with a removable lining 40 coupled to the pressure sensitive adhesive on the inner side of the bandage (i.e., the surface to be applied to the skin) Materials suitable for use as a removable liner 40 include, but are not limited to, kraft papers, polyethylene, polypropylene, polyester or composites of any of these materials. The lining can be coated with release agents such as fluorochemicals or silicones. For example, U.S. Pat. No. 4,472,480 describes low surface energy perfluorochemical liners. The lining can be papers, polyolefin films, or polyester films coated with silicone release materials. Examples of commercially available silicone coated release papers are POLY SLIK™, silicone release papers and other silicone release papers supplied by Loparex Inc. (Willowbrook, Ill.).

### Size/Configuration/Packaging

Adhesive bandages of the present disclosure may be made in any desired configuration and size. Typically, the bandages will be generally rectangular or diamond-shaped such as disclosed, for example, in U.S. Pat. No. D495,419 (Dunshee) and U.S. Pat. No. D578,651 (Dunshee), but other shapes may be employed. Various sizes may also be employed. In some situations it may be useful to make the adhesive bandages of the present disclosure in the sizes and configurations of conventional adhesive bandages so that existing manufacturing lines can be utilized. For example, in some embodiments, rectangular adhesive bandages of the present disclosure may have dimensions of 19 mm×75 mm or 25 mm×75 mm and diamond-shaped bandages may have dimensions of 22 mm×57 mm or 28 mm×76 mm. In other situations, it may be desirable to make longer or larger bandages to hold relatively large thermal packs in order to treat larger areas of the body. Such bandages may, for example, be the size of conventional elbow/knee adhesive bandages (e.g., 57 mm×102 mm).

Adhesive bandages of the present disclosure may be packaged in many desired configurations. Preferably, they will be packaged in sterile packages. In addition, the inner side of the bandages (i.e., the surface to be applied to the skin) is preferably covered with a removable liner 40.

### Thermal Pack

Thermal packs 50 (see FIGS. 3 & 4) useful with the adhesive bandages of the present disclosure comprise a flexible sealed envelope and a thermal agent within the envelope. The envelope should be a tough material that is liquid imperious and remains pliable and intact over the operating temperature range, for example, from freezer range temperatures when used as a cold pack up to elevated temperatures when used as a heat pack. The envelope is preferably puncture resistant and may be substantially transparent or opaque as desired. Many suitable envelope materials used in currently known thermal packs may be used for the thermal packs. The envelope can be prepared, for example, from polyethylene, polyester, polypropylene, cellulose esters, cellulose ethers, nylon, polyvinyl alcohol acetals, polyvinyl chloride acetate, polystyral, methyl methacrylate, and the like. A preferred envelope material is constructed of laminate materials that are microwaveable such as the polyethylene/nylon or nylon sclair laminate disclosed in U.S. Pat. No. 4,756,311 (Francis). One suitable envelope material is 0.0254 mm biaxially oriented nylon laminated to 0.0635 mm polyethylene, which commonly available for food packaging. Most preferably, the envelope is constructed from a linear low density polyethylene/polyester laminate such as Scotchpak™ Film, which is a 0.0254 mm polyester/0.0635 mm polyethylene laminate commercially available from 3M Company, St. Paul, Minn. Two sheets of Scotchpak™ film can be peripherally sealed to each other by a mechanically strong heat seal.

The envelope can be formed and sealed around a forming mandrel, and the thermal agent can be pumped into the envelope as the envelope is formed. The top seal of a filled envelope can be made at the same time, for example, as the bottom seal for the next envelope is made.

The thermal agent may be water or any number of heat source/sink materials. Among the desired performance criteria are that the material exhibit a desirable degree of conformability throughout the useful temperature range so that it can be conformed to the injury location to provide effective therapeutic treatment. At the same time, the thermal agent should be hard enough or stiff enough that the thermal pack can be easily slid into the adhesive bandage. It is also desirable that the thermal agent be sufficiently viscous such that it does not readily spill out of the envelope if the envelope is torn or punctured. This is particularly important when treating open wounds. It is desirable, for example, that the thermal agent is viscous enough that it will not run out of a ⅛ inch tear or hole in the envelope unless it is squeezed out. Typically, the thermal agent will have a viscosity between about 30,000 cP and about 100,000 cP.

Thermal gels and pastes are often utilized in hot and cold packs and can be suitable for use in a thermal pack 50. A suitable paste comprising water, salt, cellulose and flour is described, for example, in U.S. Pat. No. 4,865,012 (Kelley). Known thermal gels include gels containing glycerine (see, for example, U.S. Pat. No. 3,780,537 (Spencer) and U.S. Patent Application Pub. No. US 2010/0274333 (Dunshee et al.)); gels containing propylene glycol (see, for example, U.S. Pat. No. 5,843,145 (Brink)); gels containing a crosslinked, water-absorbing polymer such as crosslinked polyacrylamide and sodium polyacrylate (see, for example, U.S. Pat. No. 5,697,961 (Kiamil); and hydrophilic gels prepared from starting materials such as poly(ethylene oxide), polyvinyl pyrrolidone, polyacrylamide, anionic polyacrylamide, polyvinyl alcohol, maleic anhydride-vinylether copolymers, polyacrylic acid, ethylene-maleic anhydride copolymers, polyvinylether, dextran, gelatin, hydroxyl propyl cellulose, methyl cellulose, carboxymethyl cellulose, hydroxyethyl-carboxymethyl cellulose, hydroxyethyl cellulose, propylene glycol alginate, sodium alginate, polyethyleneimine, polyvinyl alkyl pyridinium halides, polyproline, natural starches, casein, proteins, polymethacrylic acid, polyvinylsulfonic acid, polystyrene sulfonic acid, polyvinylamine, poly-4-vinylpyridine, polymerized monoesters of olefinic acids, polymerized diesters of olefinic acids, acrylamide and difunctional polymerizable materials (e.g., diacids, diesters or diamides), and the like.

Preferably, the thermal agent is a gel. Thixotropic gels are particularly useful because they are less likely to run out of the thermal pack if it becomes punctured or torn.

The above-mentioned list of suitable thermal agents is not intended to be limiting. One of skill in the art will appreciate that various thermal agents may be utilized.

The thermal pack 50 can be made in any desired size or shape that fits snuggly within the pocket formed by the absorbent pad of the adhesive bandage. Typically the thermal pack 50 will be rectangular in shape although other shapes are possible. Surprisingly, it has been found that therapeutic benefit/pain relief can be obtained with a relatively small pack. Conventional thermal packs usually contain about 7.5 g/in² of thermal agent. In some embodiments, the thermal pack of the present disclosure contains between about 3 g/in² and about 5 g/in² of thermal agent (preferably, between about 3 g/in² and about 4 g/in² of thermal agent). In some embodiments, the thermal pack 50 is between about 1 inch and 2 inches wide (preferably, between about 1 inch and 1.5 inches wide) and between about 1 inch and 4 inches long (preferably, between about 2 inches and 4 inches long). The thermal packs 50 may contain relatively small amounts of thermal agent, yet still provide therapeutic benefit. For example, a 1.5 inch by 2 inch thermal pack may contain only about 10 grams to about 12 grams of thermal agent and still be effective. The thickness of the thermal agent in the pack may be, for example, from about 0.25 inches to about 0.75 inches, from about 0.25 inches to about 0.5 inches, or from about 0.3 to about 0.4 inches.

Envelopes the size of conventional condiment packets (e.g., the ketchup and mustard packets found in fast food restaurants) may be utilized. Interestingly, actual ketchup and mustard packets (i.e., packets containing ketchup or mustard) may be frozen and used as the thermal pack 50.

In other embodiments, it may be desirable to make the thermal pack 50 larger in order to treat larger areas of the body.

### Thermal Management Kit

The adhesive bandages of the present disclosure can be provided in a thermal management kit containing one or more of the adhesive bandages and one or more thermal packs. The adhesive bandages may be used as conventional medical adhesive bandages (that is, without the thermal pack) or they may be used for thermal treatment. To use the thermal management kit of the present disclosure, a user can adhere the adhesive bandage to the area of the body requiring treatment and then slide the thermal pack into the pouch on the bandage. Alternatively, the user can insert the thermal pack into the bandage and then adhere the bandage containing the thermal pack to the area of the body requiring treatment. If the user wants to extend treatment (for example, cooling) after the thermal pack has become too warm, the user can easily slide out the used thermal pack and insert a new thermal pack without removing or replacing the bandage.

The complete disclosures of the publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and set forth herein and that such embodiments are presented by way of example only with the scope of the invention intended to be limited only by the claims set forth herein as follows.

1. A dual purpose adhesive bandage comprising:
(a) a backing,
(b) a pressure sensitive adhesive disposed on the backing, and
(c) an absorbent pad adhered to the backing by the pressure sensitive adhesive; wherein
the absorbent pad is configured so as to form a pouch having at least one open end for releasably receiving a thermal pack.

2. The adhesive bandage of claim 1 wherein the absorbent pad is folded to form a tube.

3. The adhesive bandage of claim 1 wherein the absorbent pad is folded in a c-fold.

4. The adhesive bandage of claim 1 wherein the absorbent pad comprises cotton gauze laminated to netting.

5. The adhesive bandage of claim 1 wherein the absorbent pad comprises a nonwoven material.

6. A thermal management kit comprising:
(a) an adhesive bandage comprising a backing, a pressure sensitive adhesive disposed on the backing, and an absorbent pad adhered to the backing by the pressure sensitive adhesive, wherein the absorbent pad is configured so as to form a pouch having at least one open end for releasably receiving a thermal pack; and
(b) a thermal pack comprising a flexible sealed envelope and a thermal agent within the envelope.

7. The thermal management kit of claim 6 wherein the thermal pack is a cold pack.

8. The thermal management kid of claim 6 or 7 wherein the thermal agent is a gel.

9. The thermal management kid of claim 8 wherein the thermal agent is a thixotropic gel.

10. The thermal management kit of claim 6 wherein the thermal pack contains between about 3 g/in² and about 5 g/in² of thermal agent.

11. The thermal management kit of claim 10 wherein the thermal pack contains between about 3 g/in² and about 4 g/in² of thermal agent.

12. The thermal management kit of claim 6 wherein the thermal pack is from about 1 inch to about 2 inches wide and from about 1 inch to about 4 inches long.

13. The thermal management kit of claim 12 wherein the thermal pack is from about 1 inch to about 1.5 inches wide and from about 2 inches to about 4 inches long.

14. The thermal management kit of claim 6 wherein the thermal pack contains from about 10 grams to about 30 grams of thermal agent.

15. The thermal management kit of claim 14 wherein the thermal pack contains from about 10 grams to about 12 grams of thermal agent.

16. The thermal management kit of claim 6 wherein the thickness of the thermal agent is from about 0.25 inches to about 0.5 inches.

17. The thermal management kit of claim 16 wherein the thickness of the thermal agent is from about 0.3 inches to about 0.4 inches.

18. The thermal management kit of claim 6 wherein the thermal pack is a conventional fast food ketchup or mustard packet.
